(19) United States
(12) Patent Application Publication
(10) Pub. No.: US 2012/0010470 A1
(43) Pub. Date: Jan. 12, 2012

(54) DEVICES AND METHODS FOR ANCHORING A TUBE

(75) Inventors: Richard W. Ducharme, Winston-Salem, NC (US); John Crowder Sigmon, JR., Greensboro, NC (US); Andres F. Aguirre, Chicago, IL (US); Tyler Evans McLawhorn, Winston-Salem, NC (US); Michelle D. Martinez, Winston-Salem, NC (US); Vihar C. Surti, Winston-Salem, NC (US)

(73) Assignee: COOK MEDICAL TECHNOLOGIES LLC, BLOOMINGTON, IN (US)

(21) Appl. No.: 13/174,972

(22) Filed: Jul. 1, 2011

Related U.S. Application Data
(60) Provisional application No. 61/361,769, filed on Jul. 6, 2010.

(51) Int. Cl. A61B 1/32

(52) U.S. Cl. 600/201

(57) ABSTRACT

Devices and methods are provided for accessing a bodily opening that, among other things, are safe, reliable and repeatable. One embodiment of a device for anchoring to tissue includes an elongated tube and a magnet. The elongated tube has a proximal end opposite a distal end, and defines a central axis. The magnet is attached to the distal end of the elongated tube and has a magnetic field attractive to the magnetic material in the tissue. The magnet has an annular shape defining an aperture through which the elongated tube extends.

### CROSS-REFERENCE TO RELATED APPLICATIONS

This application claims the benefit of U.S. Provisional Application Ser. No. 61/361,769 filed on Jul. 6, 2010, entitled “DEVICES AND METHODS FOR ANCHORING A TUBE”, and application Ser. No. 13/029,295 filed on Feb. 17, 2011, entitled APPARATUS AND METHOD FOR ENDOSCOPIC SUBMUCOSAL DISSECTION” the entire contents of both of which are incorporated herein by reference.

### FIELD

The present invention relates generally to devices and methods for anchoring a tube, e.g. an overtube to provide access to a bodily opening through a bodily lumen, such as for an endoscope and other medical devices.

### BACKGROUND

Openings in bodily walls may be formed to gain access to adjacent structures of the body, such techniques being commonly referred to as translumenal procedures. For example, culdoscopy was developed over 70 years ago, and involves transvaginally accessing the peritoneal cavity by forming an opening in the cul de sac. This access to the peritoneal cavity allows medical professionals to visually inspect numerous anatomical structures, as well as perform various procedures such as biopsies or other operations, such as tubal ligation. Many transluminal procedures for gaining access to various body cavities using other bodily lumens have also been developed. For example, the bodily lumen(s) of the gastrointestinal tract are often endoscopically explored and can be utilized to provide access to the peritoneal cavity and other body cavities, all in a minimally invasive manner. U.S. patent application Ser. No. 12/025,985 filed Feb. 5, 2008, discloses such a procedure, and is incorporated herein by reference in its entirety.

Although transluminal procedures are minimally invasive, there are also various risks involved. For example, when an opening is formed in a bodily wall of the gastrointestinal tract, such as in the stomach or intestines, spillage of the stomach contents, intestinal contents or other bodily fluids into the adjacent body cavity can occur. Travel of bacteria laden fluids outside of the gastrointestinal tract may cause unwanted and sometimes deadly infection.

### BRIEF SUMMARY

The present invention provides devices and methods for anchoring to tissue having a magnetic material embedded in the tissue. One embodiment of a device for anchoring to tissue includes an elongated tube and a magnet. The elongated tube has a proximal end opposite a distal end, and defines a central axis. The magnet is attached to the distal end of the elongated tube and has a magnetic field attractive to the magnetic material in the tissue. The magnet has an annular shape defining an aperture through which the elongated tube extends.

According to more detailed aspects of the device, the magnet projects radially away from an outer surface of the elongated tube. The magnet preferably has a thickness that is greater than a thickness of the tubular wall. An end surface of the distal end of the elongated tube defines a distal plane, and the magnet includes a distal surface that is preferably positioned generally parallel to the distal plane. In one embodiment, the distal surface of the magnet is coplanar with the distal plane, and in another embodiment, the magnet is spaced proximally away from the distal plane. When the magnet is spaced away from the distal end of the elongated tube, the distal end projects into the tissue when the magnet is mated with the magnetic material in the tissue. The distal end of the tube can press against the tissue to form a fluidic seal when the magnet is mated with the magnetic material in the tissue.

In one embodiment of a method for anchoring to tissue, the method includes providing a device such as those described herein. A solution that has a magnetic material is injected into the tissue to form blebs of solution within the tissue. The device is positioned adjacent the blebs such that the magnet is magnetically coupled to the magnetic material in the tissue. According to further aspects of the methods, the positioning step preferably includes forming a fluid seal between the tube and the tissue. The method also preferably includes the step of forming an opening in the tissue. The distal end of the tube may be positioned within the opening in the tissue. The step of forming an opening in the tissue may be performed after the magnet is magnetically coupled to the magnetic material in the tissue. The step of closing the opening in the tissue may be performed while the magnet is magnetically coupled to the magnetic material in the tissue, thus providing support to the tissue for closure of the opening. The step of closing the opening may include passing a needle through the tissue from a second side of the tissue and delivering a closing device on a first side of the tissue while the magnet is magnetically coupled to the magnetic material on the first side of the tissue. The magnet of the device is decoupled from the magnetic material, and the opening in the tissue is closed using the closing device.

### BRIEF DESCRIPTION OF THE DRAWINGS

FIG. 1 is a side view, partially in cross-section, of a medical system constructed in accordance with the teachings of the present invention;

FIG. 2 is a cross-sectional view of the device depicted in FIG. 1;

FIG. 3 is an end view of the device depicted in FIG. 1;

FIG. 4 is a cross-sectional view depicting a method of using the device depicted in FIGS. 1-3;

FIG. 5 is a plan view of a step in the method for using the device depicted in FIGS. 1-3;

FIG. 6-10 are cross-sectional views depicting steps in the method of using the device of FIGS. 1-3;

FIG. 11 is a cross-sectional view depicting an alternate step in the method of using the devices of FIGS. 1-3;

FIG. 12 is a cross-sectional view depicting an alternate step in the method of using the devices of FIGS. 1-3;

FIG. 13 is a cross-sectional view of an alternate embodiment of the device depicted in FIGS. 1-3;

FIG. 14 is a cross-sectional view depicting the device of FIG. 13 in a state of being connected to tissue;

FIG. 15 is a cross-sectional view of another alternate embodiment of the device depicted in FIGS. 1-3;

FIG. 16 is a cross-sectional view of yet another alternate embodiment of the device depicted in FIGS. 1-3;

### DETAILED DESCRIPTION

The terms “proximal” and “distal” as used herein are intended to have a reference point relative to the user. Specifically, throughout the specification, the terms “distal” and “distally” shall denote a position, direction, or orientation that is generally away from the user and towards a target site, and the terms “proximal” and “proximally” shall denote a position, direction, or orientation that is generally towards the user and away from a target site. Thus, “proximal” and “distal” directions, portions of a device, or bodily regions, may depend on the point of entry for the procedure (e.g., percutaneously or laparoscopically or endoscopically).

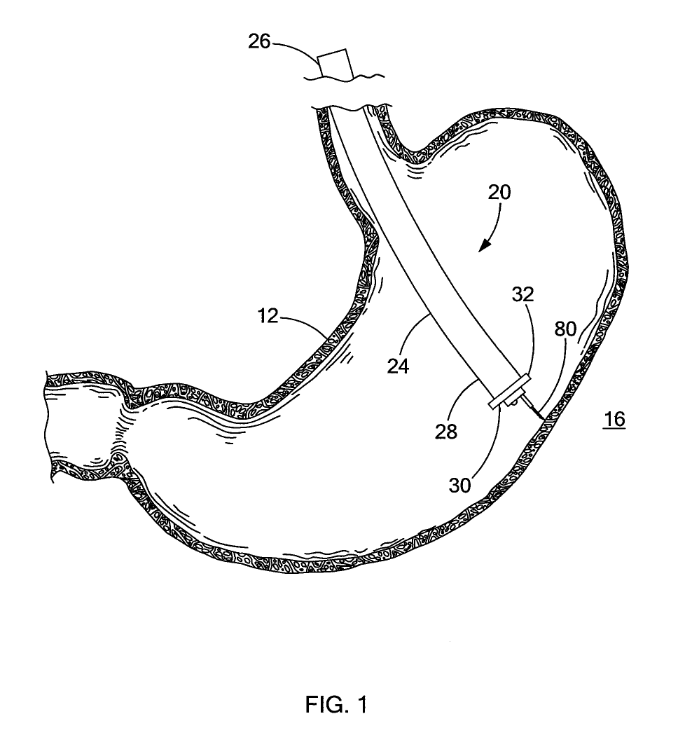

Turning now to the figures, FIG. 1 depicts a side view, partially in cross-section, of a device 20 to be anchored to tissue 12, which shown in the figure as stomach tissue. For example, the device 20 may be anchored to the tissue 12 to provide access to a bodily opening 14 (FIGS. 6-8), e.g. with an endoscope 22 or other endoscopic or laparoscopic instrument. While the opening 14 is described herein as an intentionally formed perforation in an internal bodily lumen or organ, it will be recognized by those skilled in the art that the bodily opening 14 may be unintentionally formed or naturally occurring, and may be internal or external (e.g. a laparoscopic port, ostomy, gastrostomy, etc.). The bodily opening 14 may be an opening or junction that is part of the gastrointestinal tract or other bodily lumen such as the openings at the esophageal sphincter, the pylorus sphincter, the sphincter of oddii, the ileocecal valve, or the anus.

Additionally, while the device 20 has been depicted as being used with the endoscope 30, many different medical instruments may be used in conjunction with the device 20, such as wire guides, catheters, needles, needle knives or other cutting instruments, device deployment systems, biopsy devices and the like. Similarly, the device 20 has generally be depicted as a tube, and particularly an overtube for endoscopic use, however the device may be used or adapted for use for a variety of applications, including ostomy device anchoring (e.g. colostomy bag, G-tube), wound closure, attaching a hemostatic material (e.g. gauze, submucosa, etc.), a plug or patch for perforations, stent anchoring, and the like.

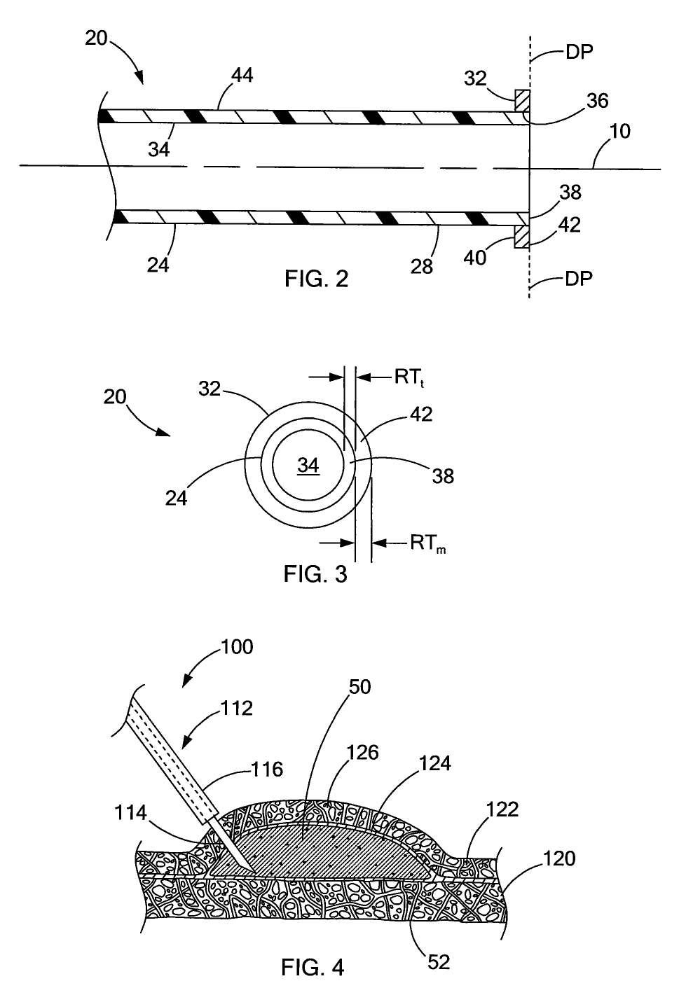

With reference to FIGS. 1 and 2, the device 20 generally comprises an elongated tube 24 extending between a proximal end 26 and a distal end 28. The elongated tube 24 is a length suitable for the particular application, in the depicted embodiment that length is suitable for use with an endoscope 30 and to traverse the significant portion of the gastrointestinal tract, e.g., from a mammalian patient's mouth through the stomach 12 and into the peritoneal cavity 16. The tube 24 may be constructed of a variety of plastic materials, such as polytetrafluorethylene (PTFE), polyethylene ether ketone (PEEK), polyamide, nylon, polyimide, polyurethane, including multi-layer or single layer constructions with or without reinforcement wires, coils or filaments. The lumen of the tube 24 is preferably sized about the same size or larger than the endoscope 30 to permit relative translation therebetween. The proximal end of the tube 24 may include a gripping surface or otherwise be attachable to the endoscope 30.

The medical device 20 also includes a magnet 32 attached to the distal end 28 of the elongated tube 24, which is used for anchoring the tube 24 to the tissue 12 as will be described in further detail herein. As best seen in FIG. 2, the elongated tube 24 generally defines a lumen 34 which is sized to receive the endoscope 30 or other medical instrument, (or bodily fluids, medication and the like depending on the application of the device 20). The tube 24 and lumen 34 generally define a central axis 10 of the medical device 20. The magnet 32 is an annular shaped body that defines an aperture 36 through which the distal end 28 of the tube 24 extends. The magnet 32 and tube 24 are preferably concentrically aligned about the central axis 10.

The distal end 28 of the tube 24 also defines an end surface 38, which in turn defines a distal plane DP as shown by the dotted lines in FIG. 2. The annular shaped magnet 32 includes a proximal surface 40 facing proximally and a distal surface 42 facing distally. The distal surface 42 of the magnet 32 is preferably positioned generally parallel to the distal plane DP, and in the depicted embodiment is coplanar with the distal plane DP. As best seen in FIGS. 2-3, the magnet 32 projects radially away from the outer surface 44 of the elongated tube 24. Preferably, the magnet 32 has a radial thickness RTₘ that is greater than a thickness of the tubular wall 24, denoted by RTₜ.

In order to anchor the device 20 to the tissue 12, a special injectable solution 50, namely a ferromagnetic gel, is utilized at the target site of the tissue 12. The injectable solution 50 is a pharmaceutically acceptable solution for use in humans and animals that has minimal tissue reactivity. The preferred viscosity for the injectable solution 50 is between about 100 to 500 mPa·S, although other viscosities may be used, and allows for blebs 126 to be formed in the tissue 12, as shown in FIG. 4, while preventing undesirable migration of the solution 50 so that the device 20 may be anchored thereto for the desired period of time. In some embodiments, the injectable solution 50 has a viscosity greater than about 100 mPa·S, and in some embodiments, a viscosity greater than about greater than about 200 mPa·S or greater than about 250 mPa·S. Non-limiting examples of suitable materials for inclusion in the injectable solution include methylcelluloses, such as carboxymethyl cellulose (CMC) and hydroxyypropyl methylcellulose (HPMC), extracellullar matrix proteins, elastin, collagen, gelatin, fibrin, agarose, and alginate or mixtures thereof. The injectable solution with be described with reference to CMC although one skilled in the art will understand that other suitable materials may also be used to form the injectable solution.

Suitable concentrations of the CMC for the injectable solution include about 1% to 10% CMC (e.g. about 1%, 1.5, 2%, 2.5%, 3%, 3.5%, 4%, 4.5%, 5%, 5.5%, 6%, 6.5%, 7%, 7.5%, 8%, 8.5%, 9%, 9.5%, or 10%). Preferably CMC concentrations range from about 2% to 5%, and more preferably about 3%. The CMC may be mixed with sterile water, saline or other pharmaceutically acceptable solution to provide a suitable concentration for injection. (CMC may be purchased from Sigma Aldrich, St. Louis, Mo.) The injectable solution 50 also includes ferromagnetic or magnetic particles combined with the CMC solution for injection into the patient. The ferromagnetic particles may be made from the following non-limiting list of materials including nickel, cobalt, or iron, alloys thereof, stainless steel, and including rare earth magnet alloys such as a neodymium-iron-boron alloy or a samarium-cobalt alloy and the like. The ferromagnetic particles may be mixed with the CMC solution so that the ferromagnetic particles become encapsulated by the CMC and prevent accumulation of the ferromagnetic particles just past the tip of the injection needle. By way of non-limiting example, a 1-3% CMC solution mixed with water may be combined with ferromagnetic particles having 1-10× the weight of the CMC powder. Preferably the CMC to ferromagnetic particle ratio is 8:1 to 1:1 by mass. Other concentrations of CMC and ferromagnetic particles may also be used. Further details of the injectable solution 50 may be found in co-pending U.S. application Ser. No. 13/029,295 filed Feb. 17, 2011, the disclosure of which is hereby incorporated by reference in its entirety.

A method for anchoring the device 20 to tissue 12 will now be described with reference to FIGS. 4-8. In FIG. 4, a delivery device 100 is shown delivering the injectable solution to a tissue treatment site 110. A distal portion 112 of the delivery device 100 is shown in FIG. 4 including an inner shaft 114 extending out of an outer catheter 116 so that the inner shaft 114 extends into the tissue 12. The inner shaft 114 may be a needle, cannula or other elongate tubular structure suitable for insertion into the tissue 12. The inner shaft 114 is inserted between a first layer of tissue 120 and a second layer of tissue 122. The layers 120, 122 may be any adjacent layers of tissue, for example, the muscularis and submucosal layers. As shown in FIG. 4, the injection of the solution 50 between the first layer 120 and the second layer 122 forms a fluid-filled pocket 124 that forces separation between the first and second layers 120, 122, breaking the attachments between the tissue layers 120, 122, and forming an elevated tissue portion or bleb 126. The injection of the solution 50 may also occur within a single layer of tissue as will be recognized by those skilled in the art.

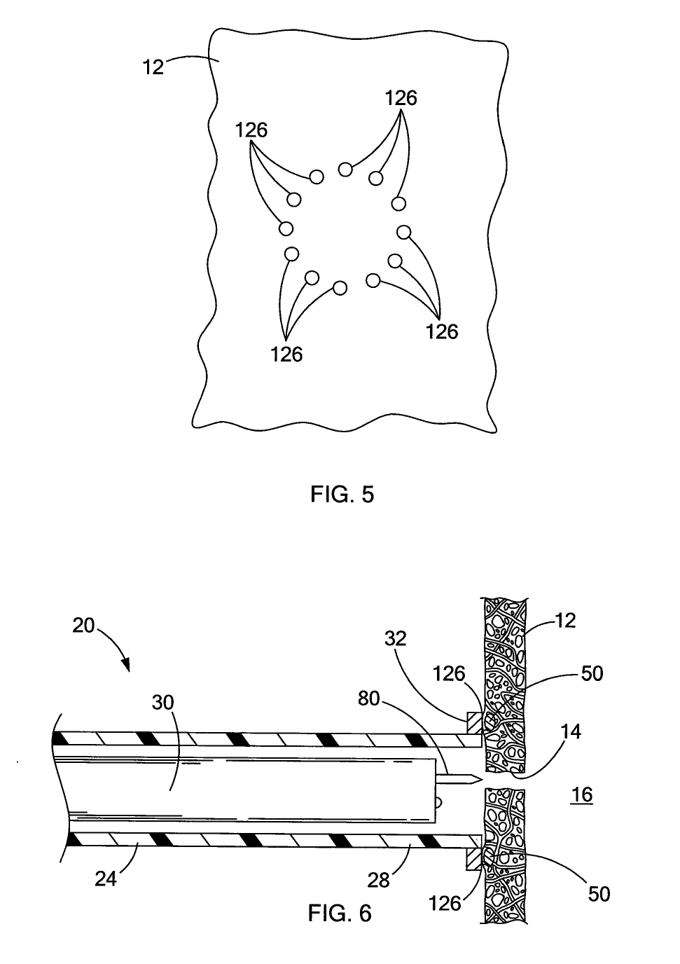

Turning now to FIG. 5, a series of blebs 126 may be formed in the tissue 12, the series preferably formed in an annular configuration that corresponds to the annular shape of the magnet 32, as shown in the figure. Depending on the particular application or type of tissue, as well as the desired degree of anchoring force and sealing to the tissue 12, any number of blebs 126 may be formed in any shape. For example, a series of injections that are immediately adjacent each other may be formed to create a continuous annular bleb.

As best seen in FIG. 6, once the solution 50 has been injected to form the blebs 126, the device 20 may be advanced distally (to the right on a page in FIG. 6) such that the magnetic field from the magnet 32 becomes attractive to the particles 52 in the solution 50, whereby the magnet 32 engages the tissue 12 at the blebs 126 to anchor the tube 24 thereto. The magnetic attraction between the magnet 32 and ferromagnetic solution 50 resists the withdrawal (i.e. distal translation) of the overtube 24. Preferably, a fluidic seal is formed when the magnet 32 is mated with the blebs 126 in the tissue 12. As used herein the term fluidic seal means substantially impervious to fluids, but does not require that the barrier is completely leak-proof. A fluidic seal herein substantially prevents the flow of fluid or other contents through the seal at fluid pressures normally found in the body at the location of tissue 12.

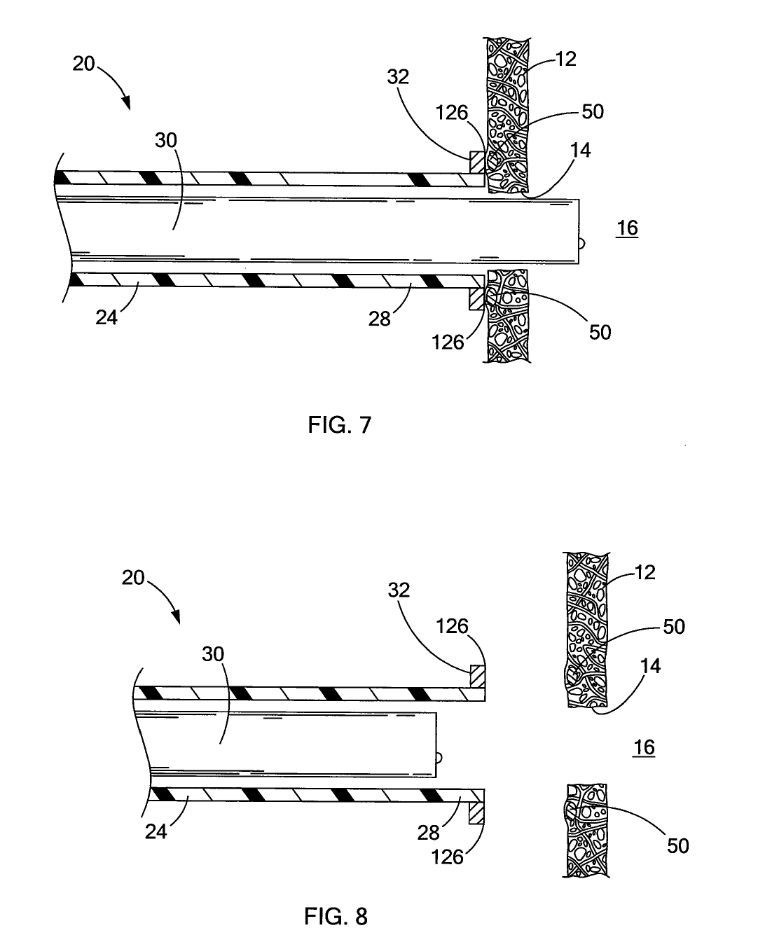

As also shown in FIG. 6, the method may also include forming an opening 14 in the tissue 12. For example, the endoscope 30 may be advanced through the tube 24 and a cutting instrument such as a needle knife 80 may be advanced through a working channel of the endoscope 30 and used to cut the tissue 12 to form the opening 14. Likewise, dilation devices such as dilation balloons and catheters may be used to enlarge the size of the opening 14 so that other medical instruments and/or the endoscope 30 may be advanced through the opening 14 and into the cavity 16 beyond the tissue 12, such as the peritoneal cavity, as shown in FIG. 7. The endoscope 30 or other medical instruments may be inserted, withdrawn and reinserted through the tube 24 to access the distal cavity 16 as many times as needed or desired. At the same time, the tube 24 maintains its access to the opening 14 while providing a fluidic seal with the tissue 12.

After the device 20 has been anchored to the blebs 126 in the tissue 12, and the endoscope 30 or other medical device has been passed through opening 14 to visualize or perform a procedure within the cavity 16, the device 20 may be detached from the tissue 12. Generally, a sufficient proximally-directed force on the proximal end 26 of the tube 24 (to the left on the page in FIG. 7) will overcome the magnetic force between the magnet 32 and the ferromagnetic particles 52 in the solution 50 forming the blebs 126. In the detached configuration shown in FIG. 8, the opening 14 in the tissue 12 may be closed.

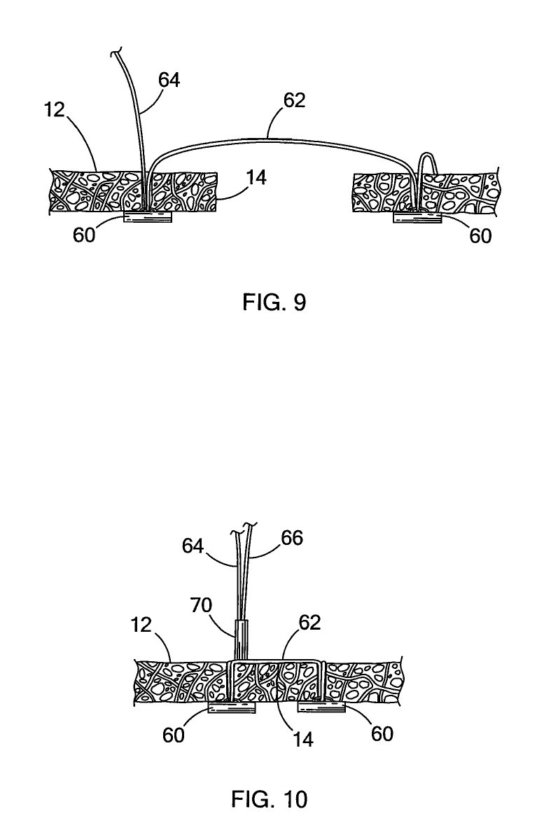

The method may also include closing the perforation 14, which in accordance with the teachings present invention, includes using a delivery needle to pass a plurality of visceral anchors 60 through the bodily wall 12 adjacent the periphery of the perforation 14, as shown in FIG. 9. Exemplary suturing devices and perforation closure methods are disclosed in copending U.S. patent application Ser. Nos. 11/946,565 filed Nov. 28, 2007, 12/125,525 filed May 22, 2008, 12/191,277 filed Aug. 13, 2008, 12/191,001 filed Aug. 13, 2008, 12/348,180 filed Jan. 2, 2009, 12/548,868 filed Aug. 27, 2009, 12/770,012 filed Apr. 29, 2010, and 12/753,111 filed Apr. 2, 2010, the disclosures of which are hereby incorporated by reference in their entireties. Generally, in one embodiment the anchors 60 are sequentially positioned around the perforation 14 in a semi-annular or annular shape as shown. A suture 62 slidably passes through a loop formed in each of the anchors 60, and the ends 64, 66 of the suture 62 are then tensioned to reduce the distance between the visceral anchors 60 and compress the bodily wall 12 around the perforation 14, as depicted in FIG. 10. The ends 64, 66 of the suture 62 are secured to maintain the compression of the bodily wall 12, such as through the use of a suture lock 70. Exemplary suture locks are disclosed in U.S. patent application Ser. Nos. 12/125,525 and 12/191,001, the disclosures of which are incorporated herein by reference in their entirety. It will be recognized that any now known or future developed method for securing the ends 64, 66 of the suture 62 may be employed, such as knotting, tying, clamps, rivets and the like.

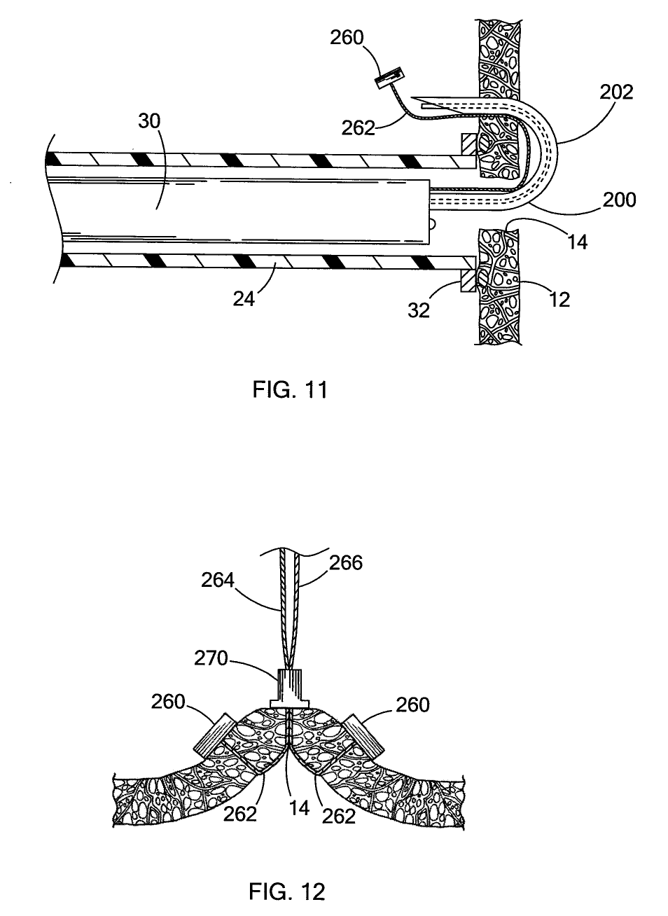

In other embodiments of closing the perforation, the device 20 may be used to support the tissue 12 to facilitate closure of the opening 14. For example, FIGS. 11 and 12 depict a flexible puncturing needle 200 projecting from the distal end 28 of the tube 24, via the endoscope 30. The flexible puncturing needle 200 includes a needle lumen having a tissue anchor 10 therein for delivery through the tissue 12 from the distal side of the tissue to the proximal side. Generally, the flexible puncturing needle 200 includes a distal portion 202 that is operable between a first linear configuration and a second non-linear configuration, e.g. the retroflexed configuration shown, using a a shape memory material such as nitinol or other similar shape memory alloys, or mechanical means such as a control wire connected to the distal end of the needle 200. As the distal end of the needle 200 is passed through the tissue 12, the device 20 and namely the tube 24 and magnet 32 support the tissue (in the distal direction) to facilitate puncturing the tissue 12 and delivery of the tissue anchors 260 connected to suture 262 to the proximal side of the tissue. The sutures 262 may be collected and tied together in any known manner, e.g. using a suture lock 270. Tension on the sutures 262 and anchors 260 causes closure of the opening 14 in the tissue 14 as shown in FIG. 12.

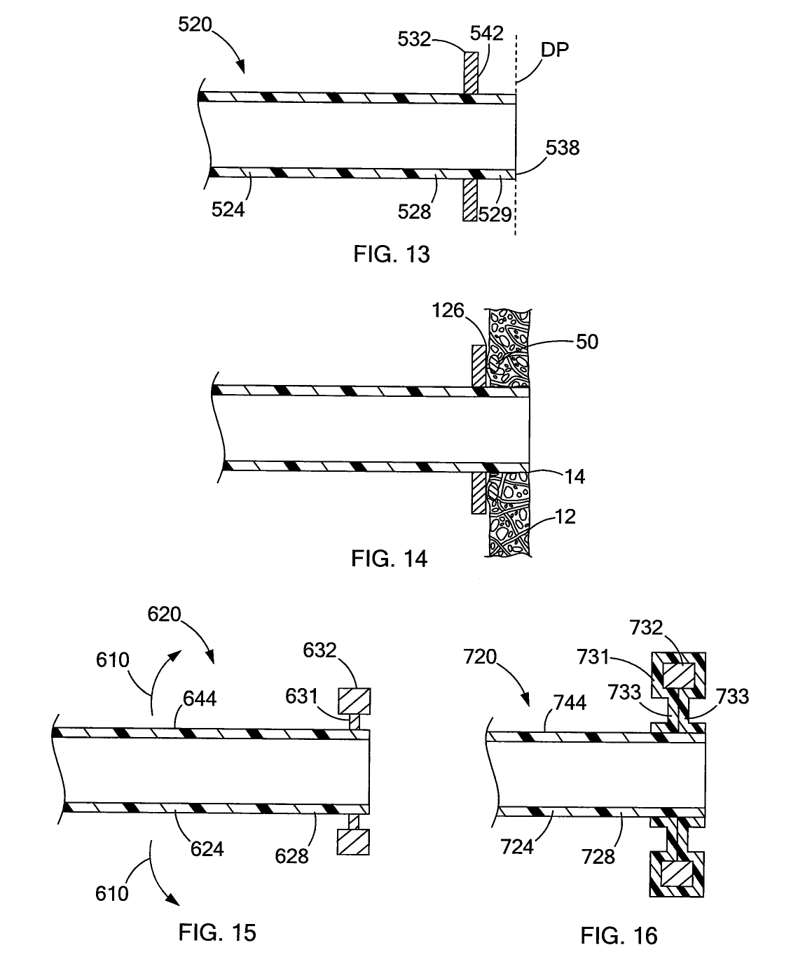

Several alternate embodiments of the device 20 will now be described with reference to FIGS. 13-16. In FIG. 13, it can be seen that the medical device 520 again includes an elongated tube 524 with a distal end 528 having a magnet 532 attached thereto. However, in this embodiment, the magnet 532 includes a distal surface 542 which is spaced proximally away from the distal plane DP defined by the end surface 538 of the tube 524. As best seen in FIG. 14, this allows the protruding distal-most portion 529 of the tube 524 (i.e. the portion extending distally beyond the magnet 532) to enter into the opening 14 of the tissue 12 when the magnet 532 is attracted and engaged with the blebs 126 formed by the solution 50. Preferably, the axial length of the distal-most portion 529 corresponds to a thickness of the tissue as shown in FIG. 14. The natural elasticity of the tissue 12 will cause the tissue to collapse around the tube 524 and provide an additional fluidic seal therewith, supplementing the seal with the tissue 12 formed between the blebs 126 of solution 50 and the magnet 532.

As shown in FIG. 15, another embodiment of the device 620 may again include a tube 624 having a distal end 628 to which an annular-shaped magnet 632 is attached. However, in this embodiment a plastic disk or ring 631 is used to connect the magnet 632 to the outer surface 644 of the tube 624. The disk 631 has sufficient flexibility to allow the tube 624 to move relative to the magnet 632, e.g. to rotate as indicated by the arrows 610 in FIG. 15. The disk 631 may be connected to the magnet 632 and the tube 624 by any known methods, including adhesives, bonding techniques, welding techniques, (e.g. plastic welding or spin welding) or using mechanical fasteners. The additional flexibility in manipulating the tube 624 after the magnet 632 is anchored to the tissue 12 may provide greater ranges for manipulating the instruments passed through the tube 624 (such as the endoscope 30), include for procedures in cavity 16 on the distal side of the tissue 12.

Like the embodiment of FIG. 15, the embodiment of the device 720 in FIG. 16 includes an elongated tube 724 having a distal end 728 with a magnet 732 attached thereto. In this embodiment, a connecting sleeve 731 is wrapped around the annular shaped magnet 732 and the free ends 733 are attached to the outer surface 744 of the tube 724, and preferably to each other, using any of the aforementioned techniques. For example, a thin plastic sheeting or other coating may be used to form the layer 731 and provide movement and flexibility of the tube 724 relative to the magnet 732. In both of the embodiments of FIGS. 15 and 16, an inner surface of the magnet is spaced radially away from an outer surface of the elongated tube. It will also be recognized by those skilled in the art that many other methods for allowing movement of the elongated tube relative to the magnet may be provided without departing from the scope of the present invention.

Accordingly, it will be seen that the medical systems, devices and methods of the present invention provide access to a bodily opening in a manner that is safe, reliable and easily repeatable. An endoscope or various other medical instruments may be repeatedly passed through an overtube to access the opening and structures on a distal side of the opening as needed. Further, the overtube is easily deployable, provides an effective fluidic seal with the tissue defining the opening, and is easily removed.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A device for anchoring to tissue having a magnetic material embedded in the tissue, the device comprising:
an elongated tube having a proximal end opposite a distal end, the elongated tube defining a central axis; and
a magnet attached to the distal end of the elongated tube, the magnet having an annular shape defining an aperture through which the elongated tube extends, the magnet having a magnetic field attractive to the magnetic material in the tissue.

2. The device of claim 1, wherein the magnet projects radially away from an outer surface of the elongated tube.

3. The device of claim 1, wherein the elongated tube is defined by a tubular wall, and wherein the magnet has a radial thickness that is greater than a thickness of the tubular wall.

4. The device of claim 1, wherein an end surface of the distal end defines a distal plane, and wherein the magnet includes a distal surface positioned generally parallel to the distal plane.

5. The device of claim 4, wherein the distal surface of the magnet is coplanar with the distal plane.

6. The device of claim 4, wherein the distal surface of the magnet is spaced proximally away from the distal plane.

7. The device of claim 1, wherein the magnet is spaced proximally away from an end surface of the distal end of the elongated tube.

8. The device of claim 9, wherein the distal end of the elongated tube projects into the tissue when the magnet is mated with the magnetic material in the tissue.

9. The device of claim 1, wherein the distal end of the tube presses against the tissue to form a fluidic seal when the magnet is mated with the magnetic material in the tissue.

10. The device of claim 1, wherein the magnet is moveably attached to the distal end of the elongated tube.

11. The device of claim 10, further comprising a rubber disk interposed between the magnet and the elongated tube.

12. The device of claim 10, further comprising a connecting sleeve extending around the magnet, the sleeve connected to the elongated tube.

13. The device of claim 10, wherein an inner surface of the magnet is spaced radially away from an outer surface of the elongated tube.

14. A method for anchoring to tissue, the method comprising:
providing a device including a tube and a magnet attached to a distal end of the tube, the magnet having an annular shape defining an aperture through which the tube extends;
injecting a solution having a magnetic material into the tissue to form blebs of solution within the tissue;
positioning the device adjacent the blebs such that the magnet is magnetically coupled to the magnetic material in the tissue

15. The method of claim 14, wherein the positioning step includes forming a fluid seal between the tube and the tissue.

16. The method of claim 14, further comprising the step of forming an opening in the tissue.

17. The method of claim 16, further comprising the step of positioning a distal end of the tube within the opening in the tissue.

18. The method of claim 16, wherein the step of forming an opening in the tissue is performed after the magnet is magnetically coupled to the magnetic material in the tissue.

19. The method of claim 16, further comprising the step of closing the opening in the tissue while the magnet is magnetically coupled to the magnetic material in the tissue.

20. The method of claim 14, wherein the magnet is positioned on a first side of the tissue, and further comprising the steps of:
passing a needle through the tissue from a second side of the tissue and delivering a closing device on the first side of the tissue while the magnet is magnetically coupled to the magnetic material in the tissue;
decoupling the magnet from the magnetic material; and
closing the opening in the tissue using the closing device.
